(19) United States
(12) Patent Application Publication
(10) Pub. No.: US 2017/0246404 A1
(43) Pub. Date: Aug. 31, 2017

(54) APPARATUS AND METHOD FOR INJECTING FLUID INTO A SUBJECT

(71) Applicant: Case.MD, Ravenna, OH (US)

(72) Inventors: Samuel Christopher Graska, Ravenna, OH (US); Justin Rudy Gleason, Ravenna, OH (US)

(21) Appl. No.: 15/441,902

(22) Filed: Feb. 24, 2017

Related U.S. Application Data
(60) Provisional application No. 62/300,202, filed on Feb. 26, 2016.

(51) Int. Cl. A61M 5/32; A61M 5/315; A45F 5/00; A61M 5/20; A45C 11/00; A45C 13/00; A61K 31/137; A61M 5/19

(52) U.S. Cl. CPC A61M 5/3298; A61K 31/137; A61M 5/31578; A61M 5/19; A61M 5/3202; A61M 5/2033; A45C 11/00; A45C 13/001; A45F 5/00; A61M 2005/2026; A45C 2011/002; A45C 2011/003; A45F 2005/002; H04B 1/3888

(57) ABSTRACT

Apparatus and method for injecting fluid into a subject includes a fluid injecting portable communication device case. The fluid injecting portable communication device case has a communication device portion. The communication device portion has a device recess that is configured to removably hold at least a portion of a portable communication device therein. A fluid injection portion has at least two fluid injection stations. Each fluid injection station is configured to interact with a fluid injector assembly, a removable trigger block, and a removable needle cover. Each fluid injector assembly includes a trigger, a firing mechanism, a plunger, and a needle.

### RELATED APPLICATION

This application claims priority from U.S. Provisional Application No. 62/300,202, filed 26 Feb. 2016, the subject matter of which is incorporated herein by reference in its entirety.

### GOVERNMENT SUPPORT

This invention was made with government support under (identify each grant number) awarded by (identify each Federal agency). The government has certain rights in the invention.

### TECHNICAL FIELD

This disclosure relates to an apparatus and method for injecting fluid into a subject and, more particularly to an apparatus and method for use of a fluid injecting portable communication device case.

### BACKGROUND

Portable injectors, such as auto injectors, are commonly used to inject medications such as, but not limited to, epinephrine, insulin, and naloxone hydrochloride to counteract such issues as, but not limited to, anaphylaxis, diabetes, and drug overdose in adults, children, and animals. For example, injectors such as epinephrine pens are carried by many people at risk for anaphylactic allergic reactions. If the user experiences the onset of anaphylaxis, they, or a third party, can press the injector into an appropriate part of their body, such as an upper arm, thigh, abdomen, or buttocks. The injector then inserts a needle and delivers the epinephrine to the user to treat the anaphylaxis, such as by a subcutaneous injection.

### SUMMARY

In an aspect, a fluid injecting portable communication device case is provided. The fluid injecting portable communication device case has a communication device portion. The communication device portion has a device recess that is configured to removably hold at least a portion of a portable communication device therein. A fluid injection portion has at least two fluid injection stations. Each fluid injection station is configured to interact with a fluid injector assembly, a removable trigger block, and a removable needle cover. Each fluid injector assembly includes a trigger, a firing mechanism, a plunger, and a needle.

In an aspect, a method of injecting fluid into a user is provided. The fluid is associated, in a storage condition, with a portable communication device case. At least a portion of a portable communication device is removably held in a device recess of a communication device portion of the portable communication device case. First and second fluid reservoirs, each containing the fluid, are provided. Each of the first and second fluid reservoirs is placed in fluid communication with a corresponding first and second needle. First and second triggers are provided. Each trigger is operatively associated with at least one of a corresponding fluid reservoir and a corresponding needle. Each fluid reservoir and the corresponding needle, in the storage condition, are fully enclosed in a corresponding first or second transport compartment of the portable communication device case. Each transport compartment is collectively formed by the communication device portion of the portable communication device case, a corresponding first or second trigger block, and a needle cover. With the first and second trigger blocks, actuation of the corresponding first and second triggers relative to the corresponding first and second fluid reservoirs is prevented when the portable communication device case is in the storage condition. The first trigger block and the needle cover are selectively removed from the first transport compartment to transform the first fluid reservoir and corresponding needle of the portable communication device case from the storage condition to a use condition. The first trigger, when the first fluid reservoir is in the use condition, is actuated. Responsive to the first trigger actuation, the first fluid reservoir and corresponding needle are moved until the needle protrudes longitudinally from the first transport compartment. Responsive to the first trigger actuation, the fluid in the first fluid reservoir is compressed to release the fluid into an ambient space from the fluid reservoir through the needle.

In an aspect, a fluid injecting portable communication device case is provided. The fluid injecting portable communication device case has a communication device portion. The communication device portion has a device recess that is configured to removably hold at least a portion of a portable communication device therein. A fluid injection portion is located on an opposite side of the communication device portion, and oppositely facing from, the device recess. The fluid injection portion includes a plurality of fluid injection stations. Each fluid injection station is configured to interact with a corresponding fluid injector assembly, a corresponding removable trigger block, and a removable needle cover. Each fluid injector assembly includes a trigger, a firing mechanism, a plunger, and a needle. Each trigger is separably operable from each other trigger. Each trigger is configured for selective longitudinal motion respective to a corresponding fluid injection station when the trigger block is removed. Each trigger is operatively connected to the corresponding firing mechanism, plunger, and needle. The needle is configured for selective longitudinal motion respective to a corresponding fluid injection station when the needle cover is removed. Each needle is moved to protrude longitudinally from the corresponding fluid injection station responsive to longitudinal force applied each trigger, transmitted longitudinally through at least the corresponding firing mechanism.

### BRIEF DESCRIPTION OF THE DRAWINGS

For a better understanding, reference may be made to the accompanying drawings, in which:

FIG. 1 is a front perspective view of a fluid injecting portable communication device according to one aspect of the present invention;

FIG. 2 is a rear perspective view of the fluid injecting portable communication device of the aspect of FIG. 1;

FIG. 3 is a rear exploded perspective view of the aspect of FIG. 1;

FIG. 4 is a cross-section taken along line 4-4 of FIG. 1;

FIG. 5 schematically illustrates example operational configurations of a portion of the aspect of FIG. 1; and

FIGS. 6-7 schematically illustrate an example sequence of operation of a portion of the aspect of FIG. 1.

### DESCRIPTION OF ASPECTS OF THE DISCLOSURE

Unless defined otherwise, all technical and scientific terms used herein have the same meaning as is commonly understood by one of skill in the art to which the present disclosure pertains.

As used herein, the term “user” can be used interchangeably to refer to an individual who prepares for, assists, and performs, and/or receives a procedure. Further, the term “user” can refer to any warm-blooded organism including, but not limited to, human beings, pigs, rats, mice, dogs, goats, sheep, horses, monkeys, apes, rabbits, cattle, farm animals, livestock, etc.

As used herein, the terms “treat,” “treating,” or “treatment” can refer to therapeutically regulating, preventing, improving, alleviating the symptoms of and/or reducing the effects of an illness, such as, but not limited to, anaphylaxis, diabetes, and drug overdose. As such, treatment also includes situations where anaphylaxis, diabetes, drug overdose, and any other similar illness, or at least a symptom associated therewith, is completely inhibited, e.g., prevented from happening or stopped (e.g., terminated) such that the subject no longer suffers from the anaphylaxis, diabetes, drug overdose, and any other similar illness, or at least the symptom(s) associated therewith.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” can be interpreted to include X and Y.

It will be understood that when an element is referred to as being “on,” “attached” to, “connected” to, etc., another element, it can be directly on, attached to, connected to, coupled with or contacting the other element or intervening elements may also be present.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

The invention comprises, consists of, or consists essentially of the following features, in any combination.

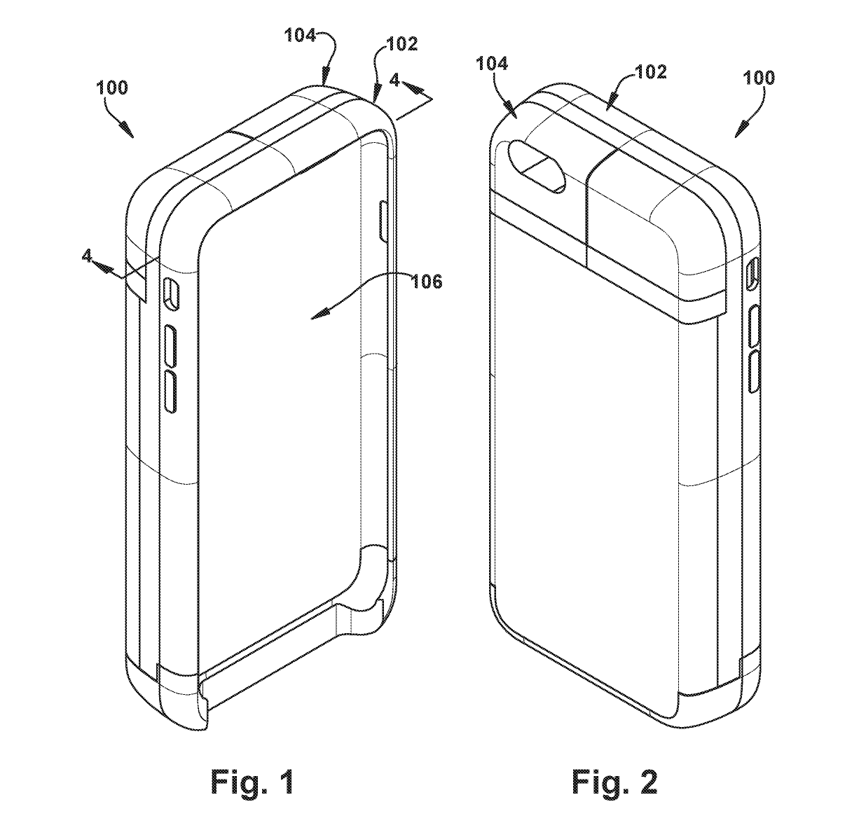

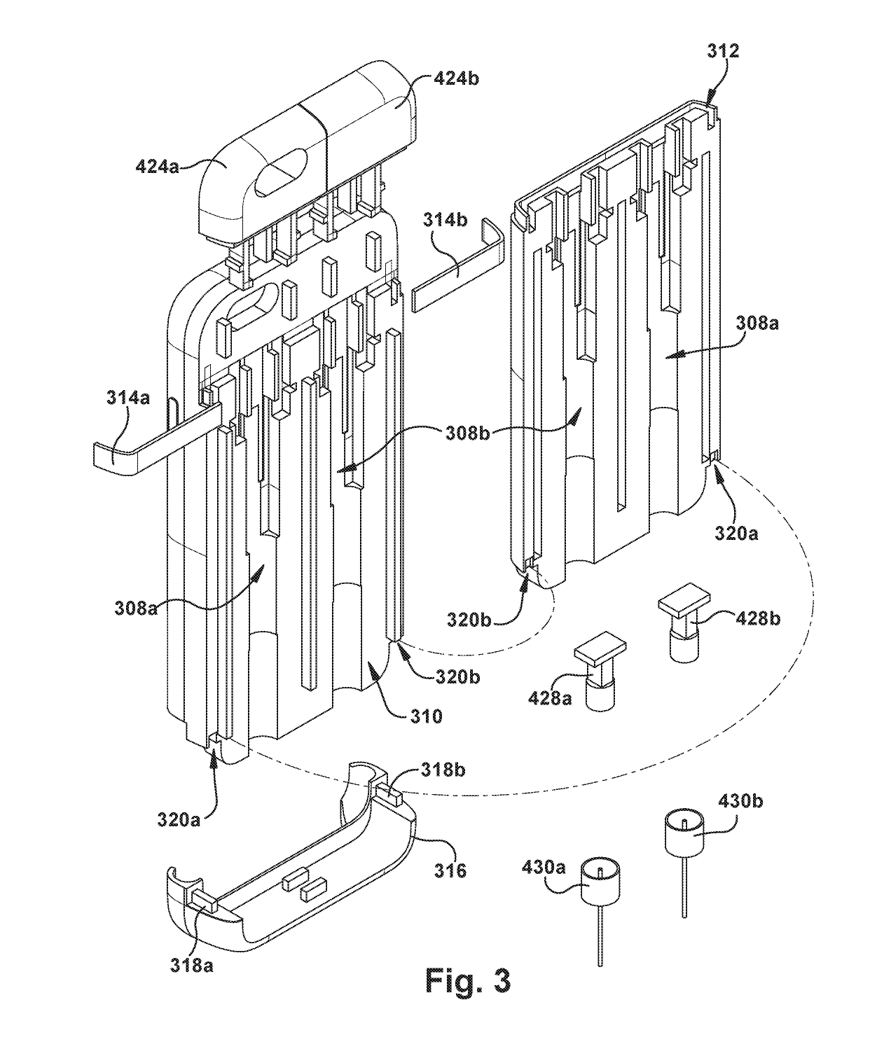

FIGS. 1-2 depict a fluid injecting portable communication device case 100. The fluid injecting portable communication device case 100 includes a communication device portion 102 and a fluid injection portion 104. The communication device portion 102 has a device recess 106 configured to removably hold at least a portion of a portable communication device (not shown) therein. The portable communication device may be a mobile phone, a smartphone, a personal digital assistant, a pager, a tablet, any portable computing device, any other suitable communication device, or any combination thereof. As shown in FIGS. 1-2, the fluid injection portion 104 is located on an opposite side of the communication device portion 102 from, and oppositely facing from, the device recess 106. In this manner, the fluid injecting features of the fluid injecting portable communication device case 100 can be provided on the “backside” of a mobile phone or other communication device, which the fluid injecting portable communication device case 100 can be configured to accept. For example, a specific fluid injecting portable communication device case 100 could be configured to accept/hold a particular make and model of smartphone.

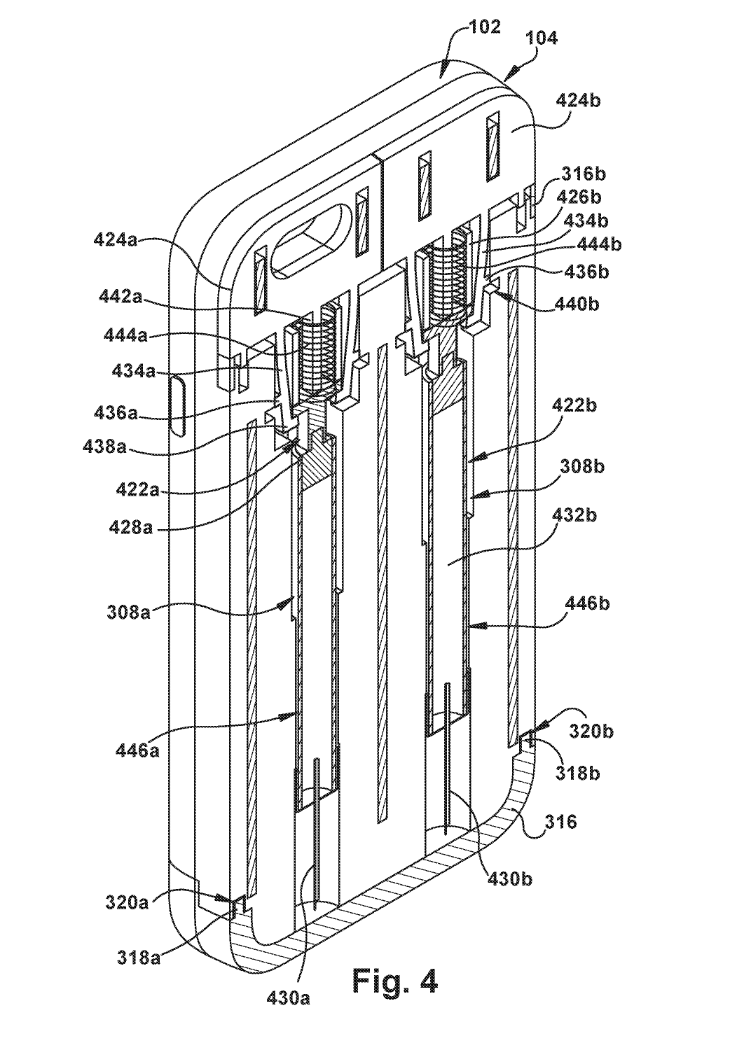

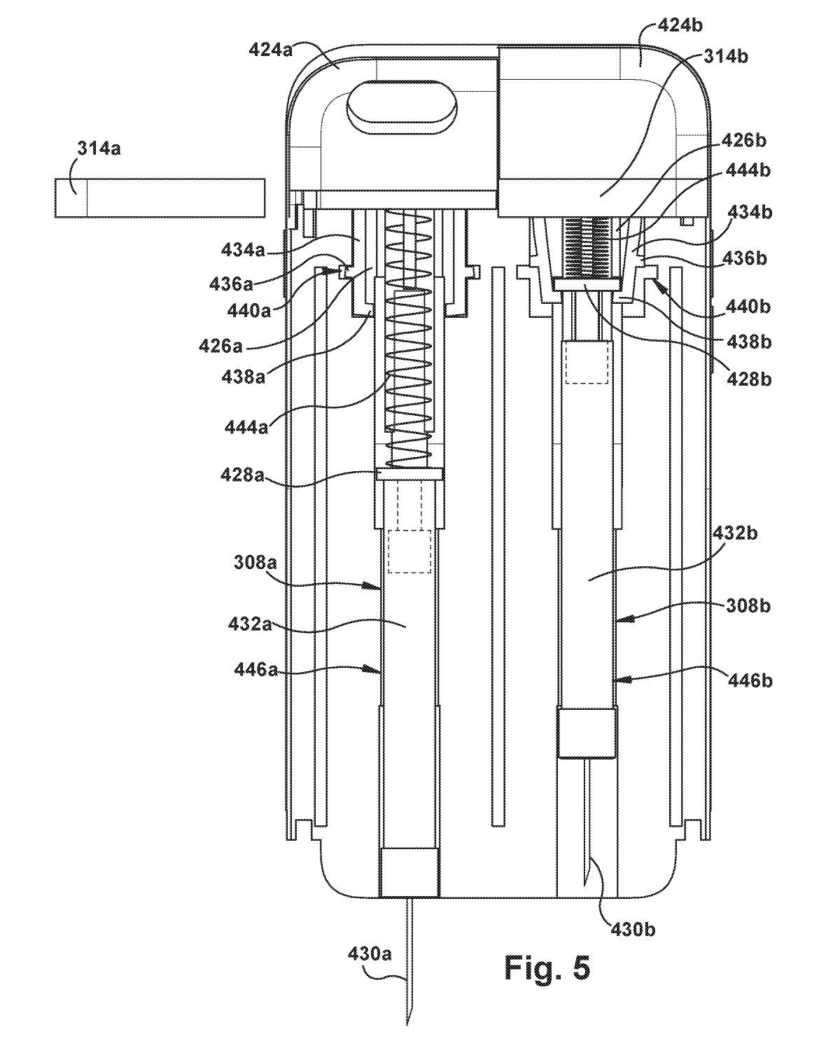

As shown in FIG. 3, the fluid injection portion 104 may have a fixed portion 310 and a selectively removable second portion in the form of a station cover 312 (depicted in an “open-book” flipped orientation in the exploded view of FIG. 3). As shown in FIGS. 3-4, the fluid injection portion 104 has at least two fluid injection stations 308, shown in FIGS. 3-4 as a first fluid injection station 308a and a second fluid injection station 308b. The station cover 312 may be configured for selective covering engagement with the fluid injection stations 308. The fluid injection stations 308 may be located on at least one of the fixed portion 310 and the station cover 312.

Each fluid injection station 308 is configured to interact with a removable trigger block 314. The trigger block 314 may be in sliding engagement with a corresponding fluid injection station 308, thereby permitting the trigger block 314 to be slidingly removed from the fluid injection portion 104. Each fluid injection station 308 may have a different corresponding trigger block 314, as shown in FIG. 3. Instead of having different corresponding trigger blocks 314, the fluid injection stations 308 may share a single trigger block (not shown).

Each fluid injection station 308 is configured to interact with a removable needle cover 316. As shown in FIGS. 3-4, the needle cover 316 may have a locking mechanism 318 that corresponds to a receiving mechanism 320 on at least one of the communication device portion 102 and fluid injection portion 104 to place the needle cover 316 in locking engagement with at least one of the communication device portion 102 and fluid injection portion 104. The locking engagement may be a snap-fit engagement, a hook and loop engagement, a press-fit frictional engagement, any other suitable engagement, or any combination thereof. A single removable needle cover 316 may be associated with the fluid injection stations 308, as shown in FIGS. 3-4. Instead of having a single removable needle cover 316, each of the fluid injection stations 308 may have a different corresponding needle cover (not shown). The needle cover 316 may also at least partially removably cover a bottom portion of the portable communication device (not shown).

As shown in FIGS. 3-4, each fluid injection station 308 is configured to interact with a fluid injector assembly 422. The fluid injector assembly 422 may be associated with at least one of the communication device portion 102, the fixed portion 310, and the station cover 312. Each fluid injector assembly 422 includes a trigger 424, a firing mechanism 426, a plunger 428, a needle 430, and a fluid reservoir 432 in fluid communication with the needle 430.

Each trigger 424 is separably operable from each other trigger 424. Each trigger 424 is configured for selective longitudinal motion respective to a corresponding fluid injection station 308 when a corresponding trigger block 314 is removed. The term “longitudinal” is used herein to indicate a substantially vertical direction, in the orientation of at least FIG. 4. Each trigger 424 is operatively associated with, and/or operatively connected to, at least one of a corresponding firing mechanism 426, plunger 428, fluid reservoir 432, and needle 430.

The firing mechanism 426 is configured to transmit longitudinal force selectively applied onto the trigger 424 to the plunger 428. The firing mechanism 426 includes at least one elastic support arm 434. The support arm 434 has at least one prong 436 and at least one support shelf 438. Each prong 436 may have a corresponding prong recess 440 in a corresponding fluid injection station 308. When the prong 436 is engaged with the prong recess 440, the support arm 434 is in a natural state. When the prong 436 is not engaged with the prong recess 440, the support arm 434 is contorted from the natural state. When contorted from the lateral state, the support arm is bent laterally inward. The term “lateral” is used herein to indicate a direction substantially perpendicular to the “longitudinal” direction, and is shown as the horizontal direction in the orientation of at least FIG. 4. The prong 436 and the support shelf 438 may extend laterally away from the support arm 434 in opposite directions.

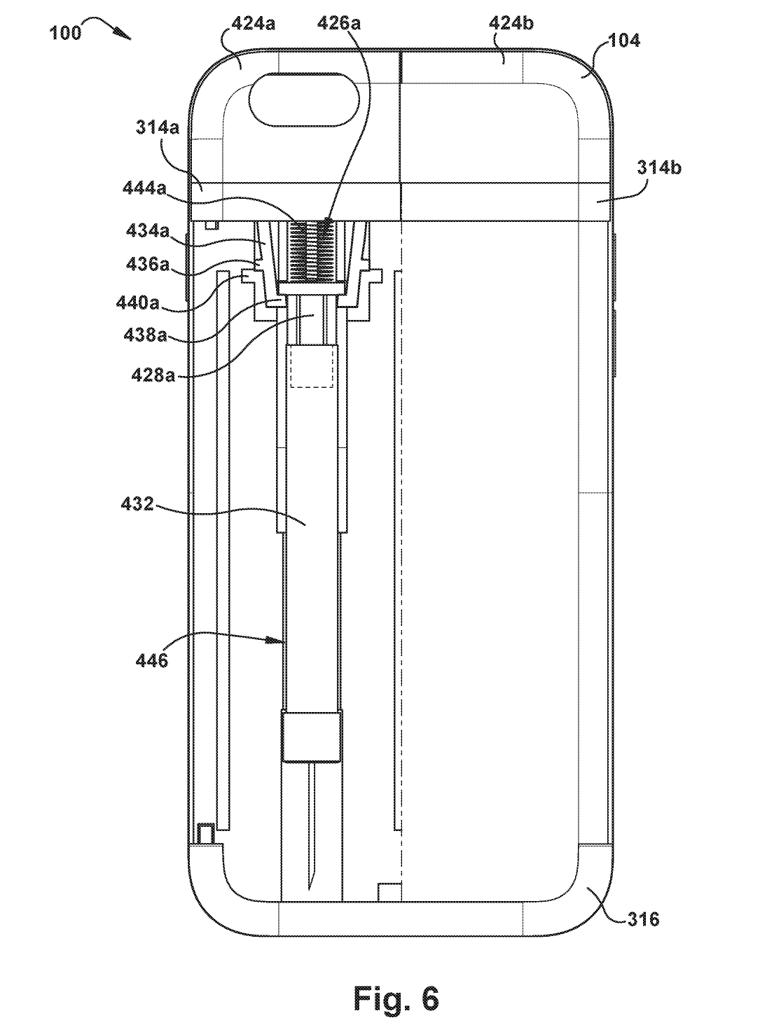

As shown in FIG. 4, each of the firing mechanisms 426 includes a rod 442 that is at least partially surrounded by a biasing member 444. The biasing member 444 may be, but is not limited to, a spring. The biasing member 444 may extend between the trigger 424 and the plunger 428. As shown in FIG. 5, the firing mechanism 426 is capable of being selectively moved between a biased condition and a firing condition. The firing mechanism 426 may be selectively moved between the biased condition and the firing condition upon selective actuation, i.e., downward longitudinal movement, of the trigger 424. In the biased condition, shown on the right side of the fluid injecting portable communication device case 100, in the orientation of FIG. 5, the biasing member 444b is compressed between the trigger 424b and the plunger 428b, the support arm 434b is laterally distorted inward from the natural state because the prong 436b is not engaged with the prong recess 440b, and the support shelf 438b is engaged with at least a portion of the plunger 428b. With the plunger 428b engaged by the support shelf 438b, the plunger 428b is restricted from longitudinally moving downward, thereby restricting the biasing member 422b from expanding.

When the firing mechanism 426 is in the firing condition, shown on the left side of the fluid injecting portable communication device case 100, in the orientation of FIG. 5, the prong 436a is engaged with the prong recess 440a, which causes the support arm 434a to return to the natural state. As the support arm 434a returns to the natural state, the support shelf 438a is laterally moved in a direction opposite to the plunger 428a to disengage from the plunger 428a. Once the plunger 428a is disengaged, the plunger 428a no longer restricts the biasing member 444a from expanding, and the biasing member 444a is able to expand to longitudinally move the plunger 428a downward.

The needle 430 is configured for selective longitudinal motion respective to a corresponding fluid injection station 308 when the needle cover 316 is removed. As shown in FIG. 5, each needle 430 may be moved to protrude longitudinally from a corresponding fluid injection station 308 responsive to a longitudinal force applied to each trigger 424 and transmitted longitudinally through at least the corresponding firing mechanism 426. That is, when a longitudinal force is applied to one of the triggers 424, the longitudinal force is transmitted longitudinally to a corresponding firing mechanism 426, causing the firing mechanism 426 to move from the biased condition to the firing condition. The transition of the firing mechanism 426 from the biased condition to the firing condition drives the plunger 428 longitudinally downward to longitudinally move a corresponding needle 430 downward until the needle 430 protrudes at least partially from a corresponding fluid injection station 308.

The fluid reservoir 432 may contain a fluid, such as, but not limited to, at least one of a pharmaceutical fluid and a therapeutic fluid. The term “pharmaceutical” is defined herein as a medicinal drug. The term “therapeutic” is defined herein as of, relating to, or used in the treatment of disease or disorders by remedial agents or methods. The fluid reservoir 432 may contain, but is not limited to, epinephrine, insulin, or naloxone hydrochloride. Each fluid reservoir 432 of the fluid injecting portable communication device case 100 may be filled with a different fluid. For example, a first fluid reservoir 432a may contain insulin, and a second fluid reservoir 432b may contain epinephrine. The fluid reservoir 432 may be a pre-filled vial purchased from a pharmaceutical, or any other appropriate, company. The user may pre-fill the fluid reservoir 432 prior to use. The fluid reservoir 432 and needle 430 may be formed as a single piece. The needle 430 may be removably attached to the fluid reservoir 432. A particular fluid injecting portable communication device case 100 may be configured for use with one or more specific types of vials. A particular fluid injecting portable communication device case 100 may include a vial adapter (not shown) to accommodate one or more specific types of vials.

The plunger 428, fluid reservoir 432, and needle 430 may be a part of the same system purchased from a pharmaceutical, or any other appropriate, company. The plunger 428 may extend at least partially into the fluid reservoir 432. The plunger 428 may be in sliding engagement with the fluid reservoir 432. As described above, when the trigger 424 is actuated, the firing mechanism 426 is moved from the biased condition to the firing condition, thereby causing the plunger 428 to longitudinally move downward. As shown in FIG. 5, the longitudinal downward movement of the plunger 428 not only causes a corresponding needle 430 and fluid reservoir 432 to longitudinally move downward, but also causes the plunger 428 to move longitudinally downward within the fluid reservoir 432 to compress the fluid therein to release the fluid in the fluid reservoir 432 through the needle 430.

With reference back to FIG. 4, each of the fluid reservoirs 432 may be selectively fully enclosed in a transport compartment 446, such as a first and a second transport compartment 446a, 446b. Each transport compartment 446 is at least partially collectively formed by the communication device portion 102, the removable trigger block 314, and the removable needle cover 316. Each transport compartment 446 may fully enclose at least one of a corresponding fluid reservoir 432 and needle 430, when in the fluid reservoir 432 and the needle are in a storage condition. The storage condition is discussed in detail below. When the fluid reservoir 432 and the needle 430 are in a use condition, the fluid reservoir 432 and the needle 430 may be capable of longitudinally sliding within the transport compartment 446. The use condition is discussed in detail below.

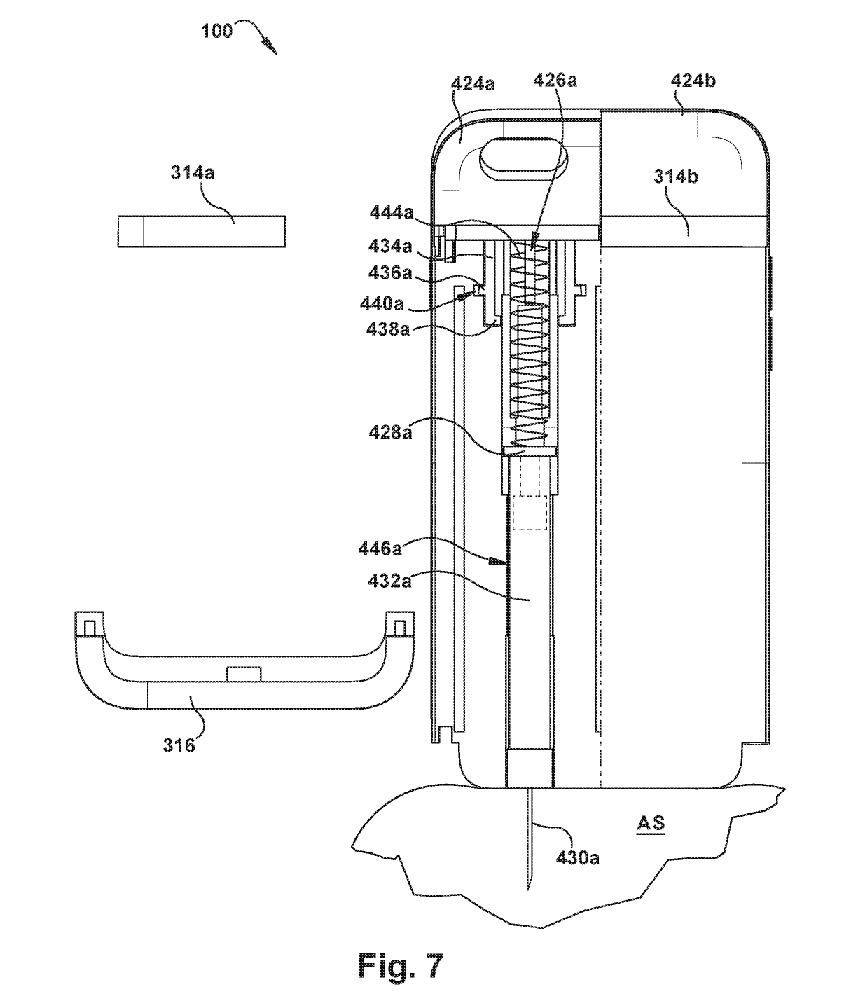

As shown in the partial cutaway view of FIG. 6, when the fluid reservoir 432 and the needle 430 are in the storage condition, each of the fluid reservoirs 432, and the corresponding needles 430 are fully enclosed in a corresponding first or second transport compartment 446a, 446b of the fluid injecting portable communication device case 100, and the first and second trigger blocks 314a, 314b are used to prevent the actuation of the corresponding first and second triggers 424a, 424b relative to the corresponding first and second fluid reservoirs 432a, 432b. By preventing the actuation of the trigger 424, the trigger block 414 prevents the trigger 424 from being longitudinally moved downward to move the firing mechanism 426 from the biased condition to the firing condition. When the fluid reservoir 432 and the needle 430 are in the use condition, as shown in FIG. 7, the trigger block 314 is removed from the transport compartment 446 and the trigger 424 is capable of being actuated. Further, when the fluid reservoir 432 and the needle 430 are in the use condition, the needle cover 316 may be removed from the transport compartment 446 to allow the needle 430 to longitudinally move downward.

In use, the fluid injecting portable communication device case 100, as described above, is provided to the user. The user removably holds at least a portion of a portable communication device (not shown) in the device recess 106 of the communication device portion 102 of the fluid injecting portable communication device case 100. The first and second fluid reservoirs 432a, 432b, each containing a fluid, are provided. Each of the first and second fluid reservoirs 432a, 432b is placed in fluid communication with a corresponding first and second needle 430a, 430b.

Although only a first fluid injection station 308a of the fluid injecting portable communication device case 100 is visible in FIG. 6 (indicated by element numbering including an “a”), each of the firing mechanisms 426 are moved into the biased condition by moving the corresponding support arms 434 to a position where the corresponding prongs 436 are not engaged with the corresponding prong recesses 440. With the prongs 436 not engaged with the prong recesses 440, the support arms 434 are contorted laterally inward to cause the corresponding support shelves 438 to engage and restrict the corresponding plungers 428 from longitudinally moving downward. With the plungers 428 engaged and restricted by the support shelves 438, the corresponding biasing members 444 are compressed between the corresponding triggers 424 and plungers 428 and restricted from expanding. The fluid reservoir 432 and the needle 430 are moved into the storage condition by fully enclosing each of the fluid reservoirs 432 and the needles 430 in the corresponding transport compartments 446, and by attaching the corresponding trigger blocks 314 and needle cover 316 to the fluid injecting portable communication device case 100.

As shown in the partial cutaway view of FIG. 7, the first trigger block 314a and the needle cover 316 are selectively removed from the first transport compartment 446a to transform the first fluid reservoir 432a and corresponding needle 430a of the fluid injecting portable communication device case 100 from the storage condition to the use condition. Removing first trigger block 314a may include sliding the first trigger block 314a substantially perpendicular to a direction of trigger 424a actuation motion, such as by sliding the trigger block 314a laterally.

With the fluid injecting portable communication device case 100 in the use condition, the user may place the fluid injecting portable communication device case 100 in an appropriate position to deliver the fluid to a desired ambient space AS. The ambient space AS may be a user's flesh, such as, but not limited to, the user's thigh. For example, the user may orient the fluid injecting portable communication device case 100 to a position in which at least one of the first and second needles 430a, 430b would inject fluid into the user's thigh upon actuation of at least one of the first and second triggers 424a, 424b. The user may position the portion of the fluid injecting portable communication device case 100 that was uncovered by the needle cover 316 to be resting on the user's thigh in a perpendicular, ninety (90) degree angle. The ambient space AS may be the surrounding atmosphere when the needle is not inserted into a user's flesh.

With the first fluid reservoir 432a in the use condition, the first trigger 424a is actuated. To actuate the first trigger 424a, the user may depress the trigger 424a to cause the trigger 424a to longitudinally move downward with respect to the first fluid reservoir 432a. The longitudinal downward movement of the trigger 432a responsively causes the firing mechanism 426a to move from the biased condition to the firing condition by longitudinally moving the support arm 434a downward until the prong 436a engages the prong recess 440a. Once the prong 436a engages the prong recess 440a, the support arm 434a is able to return to its natural state. As the support arm 434a returning to its natural state, the support shelf 438a responsively disengages from the plunger 428a by laterally moving in a direction opposite to the plunger 428a. Once the plunger 428a is disengaged, the plunger 428a and biasing member 444a are unrestricted.

With the plunger 428a and biasing member 444a unrestricted, the biasing member 444a expands to cause the plunger 428a to longitudinally move downward. The downward movement of the plunger 428a responsively causes the first fluid reservoir 432a and corresponding needle 430a to longitudinally move downward until the needle 430a protrudes longitudinally from the first transport compartment 446a. The downward movement of the plunger 428a also responsively causes the fluid in the first fluid reservoir 432a to be compressed and to release the fluid into the ambient space AS from the fluid reservoir 432a through the needle 430a.

If desired, the second trigger block 314b and the needle cover 316 may be removed from the second transport compartment 446b to transform the second fluid reservoir 432b and corresponding needle 430b of the fluid injecting portable communication device case 100 from the storage condition to the use condition. With the second fluid reservoir 432b in the use condition, the second trigger 424b is actuated. In a similar sequence as described above, responsive to the second trigger 424b actuation, the second fluid reservoir 432b and corresponding needle 430b are moved until the needle 430b protrudes longitudinally from the second transport compartment 446b. Responsive to the second trigger 424b actuation, the fluid in the second fluid reservoir 432b is compressed to release the fluid into the ambient space AS from the fluid reservoir 432b through the needle 430b.

Any element of the portable communication device 100 may be at least partially formed from silicone, polyethylene, polypropylene, stainless steel, titanium, any plastic, any polymer, any biocompatible material, any other suitable material, or any combination thereof.

It is contemplated that each trigger 424, at least one corresponding support arm 434, and a corresponding rod 442 may be integrally formed as a single piece.

It is contemplated that the fluid injecting portable communication device case 100 may be reset after at least one of the needles 430 has been used. For example, after at least one of the needles 430 has been fired to protrude from the transport compartment 446, the user may retract the needle 430 back into the transport compartment 446 and may place the needle cover 316 back on the fluid injecting portable communication device case 100 to protect the user, or others, from any unintended needle related injuries. Also, when the fluid injecting portable communication device case 100 includes a removable needle 430, the user may be able to remove and discard the needle 430 after the needle 430 has been used to the user's satisfaction and place the needle cover 316 back on the fluid injecting portable communication device case 100.

It is contemplated that the fluid injecting portable communication device case 100 may assist the user in injecting a fluid into herself/himself, another person, or an animal. For example, if a user is prone to anaphylaxis, the user may carry the fluid injecting portable communication device case 100 that has a fluid reservoir 432 containing epinephrine. If the user happened to enter into anaphylaxis while carrying the fluid injecting portable communication device case 100, the user could self-inject the epinephrine fluid into his/her body, in a similar sequence as described above.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. The description of each element number whether including an “a,” “b,” or neither, is intended to describe all like elements with the same numbering. For example, the description of element 111 describes 111, 111a, and/or 111b. Any of the described structures and components could be integrally formed as a single unitary or monolithic piece or made up of separate sub-components, with either of these formations involving any suitable stock or bespoke components and/or any suitable material or combinations of materials. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

We claim:

1. A fluid injecting portable communication device case, comprising
a communication device portion having a device recess configured to removably hold at least a portion of a portable communication device therein; and
a fluid injection portion having at least two fluid injection stations, each fluid injection station being configured for interaction with a fluid injector assembly, a removable trigger block, and a removable needle cover;
each fluid injector assembly including a trigger, a firing mechanism, a plunger, and a needle.

2. The fluid injection portable communication device case of claim 1, wherein each fluid injection station has a different corresponding trigger block.

3. The fluid injection portable communication device case of claim 1, wherein a single removable needle cover is associated with at least two fluid injection stations.

4. The fluid injection portable communication device case of claim 1, wherein the fluid injector assembly includes a fluid reservoir in fluid communication with the needle.

5. The fluid injection portable communication device case of claim 4, wherein the fluid reservoir contains at least one of a pharmaceutical fluid and a therapeutic fluid.

6. The fluid injection portable communication device case of claim 5, wherein the fluid reservoir contains epinephrine.

7. The fluid injection portable communication device case of claim 1, including a station cover configured for selective covering engagement with the at least two fluid injection stations.

8. The fluid injection portable communication device case of claim 7, wherein the fluid injector assembly includes a fluid reservoir in fluid communication with the needle, and wherein the fluid reservoir is selectively fully enclosed in a transport compartment collectively formed by the communication device portion, the removable trigger block, and the removable needle cover.

9. The fluid injection portable communication device case of claim 1, wherein the firing mechanism is configured to transmit longitudinal force selectively applied onto the trigger to the plunger.

10. The fluid injection portable communication device case of claim 9, wherein the firing mechanism includes a biasing member, and an elastic support arm, the elastic support arm includes at least one prong and at least one support shelf, and wherein each of the fluid injection stations includes a prong recess for receiving a corresponding prong.

11. The fluid injection portable communication device case of claim 10, wherein the firing mechanism is capable of being selectively moved between biased and firing conditions such that in the biased condition the biasing member is compressed between the trigger and the plunger, the prong is not engaged with the prong recess, and the support shelf is engaged with at least a portion of the plunger to restrict the plunger from longitudinally moving downward and biasing member from expanding, while in the firing condition, the prong is engaged with the prong recess, the support shelf is not engaged with the plunger, and the plunger and biasing member are unrestricted.

12. A method of injecting fluid into a user, the fluid being associated, in a storage condition, with a portable communication device case, the method comprising:
removably holding at least a portion of a portable communication device in a device recess of a communication device portion of the portable communication device case;
providing first and second fluid reservoirs, each containing the fluid;
placing each of the first and second fluid reservoirs in fluid communication with a corresponding first and second needle;
providing first and second triggers, each trigger being operatively associated with at least one of a corresponding fluid reservoir and a corresponding needle;
fully enclosing each fluid reservoir and the corresponding needle, in the storage condition, in a corresponding first or second transport compartment of the portable communication device case, each transport compartment collectively formed by the communication device portion of the portable communication device case, a corresponding first or second trigger block, and a needle cover;
with the first and second trigger blocks, preventing actuation of the corresponding first and second triggers relative to the corresponding first and second fluid reservoirs when the portable communication device case is in the storage condition;
selectively removing the first trigger block and the needle cover from the first transport compartment to transform the first fluid reservoir and corresponding needle of the portable communication device case from the storage condition to a use condition;
actuating the first trigger, when the first fluid reservoir is in the use condition;
responsive to the first trigger actuation, moving the first fluid reservoir and corresponding needle until the needle protrudes longitudinally from the first transport compartment; and
responsive to the first trigger actuation, compressing the fluid in the first fluid reservoir to release the fluid into an ambient space from the fluid reservoir through the needle.

13. The method of claim 12, wherein selectively removing the first trigger block includes sliding the first trigger block substantially perpendicularly to a direction of trigger actuation motion.

14. The method of claim 12, wherein each firing mechanism includes a biasing member and an elastic support arm, the elastic support arm including at least one prong and at least one support shelf, each of the fluid injection stations includes a prong recess for receiving a corresponding prong, and wherein the method includes
moving each of the support arms to a position where the corresponding prongs are not engaged with the corresponding prong recesses;
with the prongs not engaged with the prong recesses, moving the support arms laterally inward to cause the corresponding support shelves to engage and restrict the corresponding plungers from longitudinally moving downward;
with the plungers engaged and restricted by the support shelves, compressing the corresponding biasing members between the corresponding triggers and plungers, thereby restricting the biasing members from expanding;
actuating the first trigger by depressing and longitudinally moving the first trigger downward with respect to the first fluid reservoir;
responsive to the longitudinal movement downward movement of the first trigger, longitudinally moving the corresponding firing mechanism downward until the corresponding prong engages the corresponding prong recess;
with the prong engaging the prong recess, moving the support arm laterally outward to disengage the plunger; and
responsive to the disengagement of the plunger, expanding the biasing member to responsively cause the plunger to longitudinally move downward.

15. The method of claim 12, including:
selectively removing the second trigger block and the needle cover from the second transport compartment to transform the second fluid reservoir and corresponding needle of the portable communication device case from the storage condition to a use condition;
actuating the second trigger, when the second fluid reservoir is in the use condition;
responsive to the second trigger actuation, moving the second fluid reservoir and corresponding needle until the needle protrudes longitudinally from the second transport compartment; and
responsive to the second trigger actuation, compressing the fluid in the second fluid reservoir to release the fluid into an ambient space from the fluid reservoir through the needle.

16. The method of claim 15, wherein selectively removing the second trigger block includes sliding the second trigger block substantially perpendicularly to a direction of trigger actuation motion.

17. The method of claim 15, wherein each firing mechanism includes a biasing member and an elastic support arm, the elastic support arm including at least one prong and at least one support shelf, each of the fluid injection stations includes a prong recess for receiving a corresponding prong, and wherein the method includes
moving each of the support arms to a position where the corresponding prongs are not engaged with the corresponding prong recesses;
with the prongs not engaged with the prong recesses, moving the support arms laterally inward to cause the corresponding support shelves to engage and restrict the corresponding plungers from longitudinally moving downward;
with the plungers engaged and restricted by the support shelves, compressing the corresponding biasing members between the corresponding triggers and plungers, thereby restricting the biasing members from expanding;
actuating the second trigger by depressing and longitudinally moving the second trigger downward with respect to the second fluid reservoir;
responsive to the longitudinal movement downward movement of the second trigger, longitudinally moving the corresponding firing mechanism downward until the corresponding prong engages the corresponding prong recess;
with the prong engaging the prong recess, moving the support arm laterally outward to disengage the plunger; and
responsive to the disengagement of the plunger, expanding the biasing member to responsively cause the plunger to longitudinally move downward.

18. The method of claim 12, wherein the fluid is at least one of a pharmaceutical fluid and a therapeutic fluid.

19. The method of claim 18, wherein the fluid is epinephrine.

20. A fluid injecting portable communication device case, comprising:
a communication device portion having a device recess configured to removably hold at least a portion of a portable communication device therein;
a fluid injection portion located on an opposite side of the communication device portion, and oppositely facing from, the device recess, the fluid injection portion including a plurality of fluid injection stations, each fluid injection station being configured for interaction with a corresponding fluid injector assembly, a corresponding removable trigger block, and a removable needle cover;
each fluid injector assembly including a trigger, a firing mechanism, a plunger, and a needle;
wherein each trigger is separably operable from each other trigger, each trigger configured for selective longitudinal motion respective to a corresponding fluid injection station when the trigger block is removed, and each trigger being operatively connected to the corresponding firing mechanism, plunger, and needle;
wherein the needle is configured for selective longitudinal motion respective to a corresponding fluid injection station when the needle cover is removed; and
wherein each needle is moved to protrude longitudinally from the corresponding fluid injection station responsive to longitudinal force applied each trigger, transmitted longitudinally through at least the corresponding firing mechanism.
